(19) United States
(12) Patent Application Publication
(10) Pub. No.: US 2019/0314629 A1
(43) Pub. Date: Oct. 17, 2019

(54) METHOD OF ADIPOSE TISSUE TREATMENT

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 15/954,939

(22) Filed: Apr. 17, 2018

(51) Int. Cl. A61N 1/28; A61B 18/14; A61N 1/05

(52) U.S. Cl. CPC A61N 1/28; A61B 2018/00184; A61N 1/0502; A61B 18/1477

(57) ABSTRACT

The invention relates to a method for lipolysis using multiple RF electrodes inserted into the subcutaneous fat.

### FIELD OF THE INVENTION

The invention relates to a device in the field of fractional treatment of human tissue using RF energy.

### BACKGROUND OF THE INVENTION

Fractional devices have become common place for skin treatment. Fractional injuries to the skin and dermis can be treated by laser systems such as FRAXEL™, which sends small beams of erbium glass laser wavelength energy into the dermis, or alternatively with fractional devices, such as micro-needling, surface ablation or invasive needling. With these devices, the dermis is stimulated with an aggressive fractional trauma, which causes fractional skin resurfacing, skin tightening, or acne scar and wrinkle treatment.

U.S. Pat. No. 6,210,402 describes a method for dermatological treatment of an external body surface, applying high frequency electrical energy to the electrode terminal comprising multiple conductive elements.

U.S. Pat. Nos. 6,148,232 and 6,615,079 describe a method and device for fractional ablation of stratum corneum for transdermal drug delivery, wherein a plurality of conductive elements are applied to the stratum corneum and RF energy is applied between conductive elements.

U.S. Pat. Nos. 8,496,654 and 8,357,157 describe a device for cosmetic fractional epidermis ablation where multiple electrodes are applied to the skin surface and RF energy is applied between the multiple electrodes and a grounded return electrode, wherein the plurality of RF application elements are free of any ground electrode therebetween.

U.S. Pat. No. 8,579,896 describe fractional coagulation of skin with one electrode constructed from spaced apart elements.

U.S. Pat. No. 9,108,036 describes a skin treatment device, comprising a plurality of electrodes arranged in a cluster, and a plurality of electrodes sized substantially larger than the first size and arranged at a periphery of the cluster and spaced from the cluster, and wherein the cluster of elements are free of any portion of the larger sized electrode therebetween.

U.S. Pat. No. 9,480,836 describes a needle array penetrating into the skin and powered by a motor connecting to the array, wherein RF energy is applied between needles penetrating into the skin.

US Patent Application 20040181216 and U.S. Pat. No. 9,510,899 describe a device with needles penetrating into the dermis for collagen remodeling.

### SUMMARY OF THE INVENTION

The present invention describes a device and method for delivering radio-frequency (RF) energy in a fractional manner into the subdermal space creating thermal destruction of subcutaneous fat. A matrix of multiple conductive elements (e.g., a needle array) penetrates into a subdermal space.

RF energy can be applied between needles or alternatively a return electrode can be applied to the skin surface. Preferably, the return electrode surrounds each needle for uniform RF energy exposure. The return electrode may be large enough to avoid significant heating near the skin surface, and most of the RF energy is delivered near the needle tip inside adipose tissue.

Thickness of the dermis can vary from 0.5 mm in the periorbital area and can be as thick as 2 mm for male body areas. In order to deliver RF energy into the subcutaneous fat without significant thermal exposure to the upper layer, the electrode needles may be coated with an isolating material over the needle length longer than the thickness of the dermis. Therefore, without limitation, the coated part of the needles can be 0.5 mm or longer depending on the treatment area and the dermis thickness. For eye lid area treatment, only the top 0.5 mm of the needle may need to be coated to avoid dermis exposure, while for other body areas the coated part of the needle may be about 2 mm. If the coated part of needles has a length of 2 mm, the uncoated part of the needle will reach the fat for all types of dermis.

In one embodiment, one polarity of RF energy is applied to the multiple conductive elements while the other polarity of RF is connected to the return electrode surrounding the conductive element.

In one embodiment, the matrix of pairs of the conductive elements and the return electrodes surrounding them may get RF energy simultaneously.

In an alternative embodiment, each pair gets RF energy intermittently or one after the other. This can be important if the RF source has limited power and cannot deliver RF energy to all electrodes simultaneously.

In other embodiments, multiple conductive elements are needles with a fixed length from 0.6 mm up to 10 mm, without limitation. Alternatively, needle insertion depth can be adjusted by the user according to the dermis thickness. Needle length can be adjusted, without limitation, in the range of 0.6 mm to 10 mm manually or by using an electromechanical mechanism as a motor or solenoid. Diameter of the needle may be, without limitation, in a range of 100 microns up to 500 microns and have a sharp end. The non-coated sharp end of needles may be designed to deliver RF energy into the subcutaneous fat and may have a length from 0.1 mm up to 2 mm, without limitation.

The distance between needles may be about 1 mm or more to create strong thermal effects preferably around the needle end and avoid thermal damage in the vicinity of the return electrode.

The total area of the return electrode may be larger than the total area of the multiple conductive elements to provide strong thermal effects near each of the multiple conductive elements.

Needles used as a conductive element can be partially coated with electrically isolating material to create localized thermal effects in the vicinity of the uncoated part and protect the tissue along the coated surface.

The matrix of conductive elements penetrating into the tissue may be assembled on a single-use tip which is disposed in the end of the treatment to avoid cross-contamination.

The needles and the return electrode may be connected to the RF generator providing one or more RF voltages. The RF generator may vary RF power and pulse duration.

The device powering the applicator may comprise a microprocessor for controlling the electronics and user interface. The microprocessor may monitor one or more of the following RF parameters, including but not limited to, RF voltage, RF current, RF power, RF impedance, phase shift between RF voltage and RF current. In addition, the controller may control and monitor insertion and retraction of the conductive elements.

RF energy applied to the tip is high enough to create necrosis of adiposites in subdermal zone.

This method of treatment can be used for local reduction of fat, circumferential reduction and area contouring.

### BRIEF DESCRIPTION OF THE DRAWINGS

In the accompanying drawings:

FIG. 1 is a schematic illustration of skin structure with applied treatment tip.

FIG. 3 is a schematic illustration of the needle.

FIG. 4 is a schematic illustration of one example of replaceable tip.

### DETAILED DESCRIPTION OF THE INVENTION

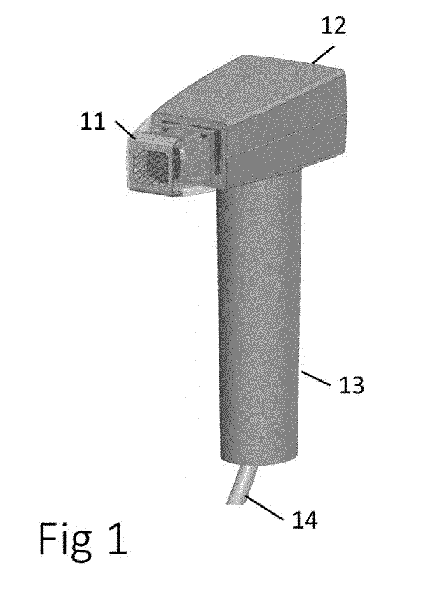

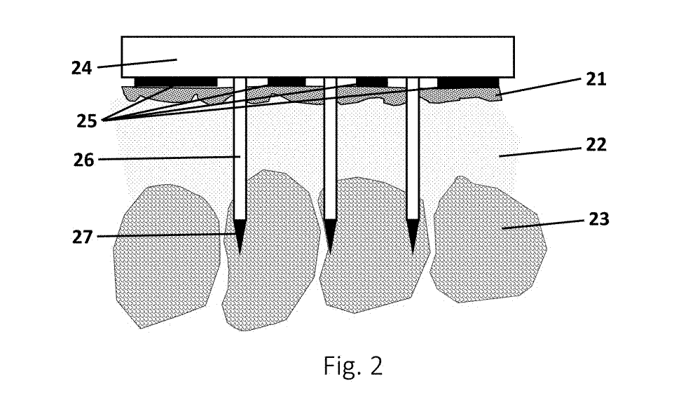

Referring first to FIG. 1, an applicator assembly comprises a housing 12 and a handle 13. A replaceable tip 11 is connected to the front side of the hand piece. RF energy and control signals are delivered to the hand piece through a cable 14.

FIG. 2 shows skin structure with a superficial layer called epidermis and having thickness of about 0.1 mm. The second layer 22 is called dermis and consists mostly of collagenous tissue. The thickness of this layer varies from 0.5 mm to 2 mm depending on gender, age and body area. The third layer is subcutaneous fat 23 which may be as thick as a few centimeters. FIG. 2 shows tip housing 24 with return electrode 25 applied to the skin surface and needles penetrating into the subcutaneous fat 23. The needles have an isolated part 26 which protects the dermis from thermal exposure. An uncoated tip 27 delivers RF energy into the subcutaneous fat.

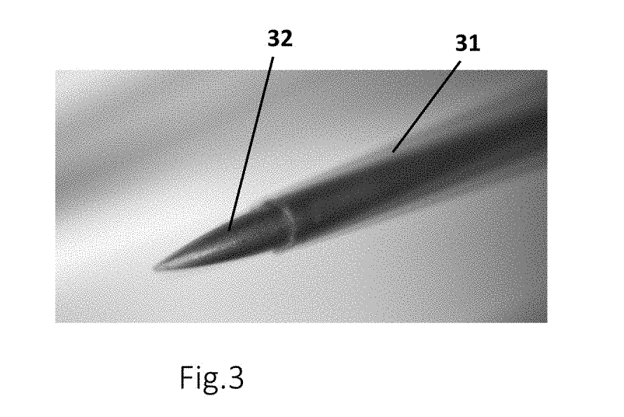

FIG. 3 shows microscope photography of the needle with a coated portion 31 and a conductive sharp tip 32 for delivering energy into the sub-dermal area.

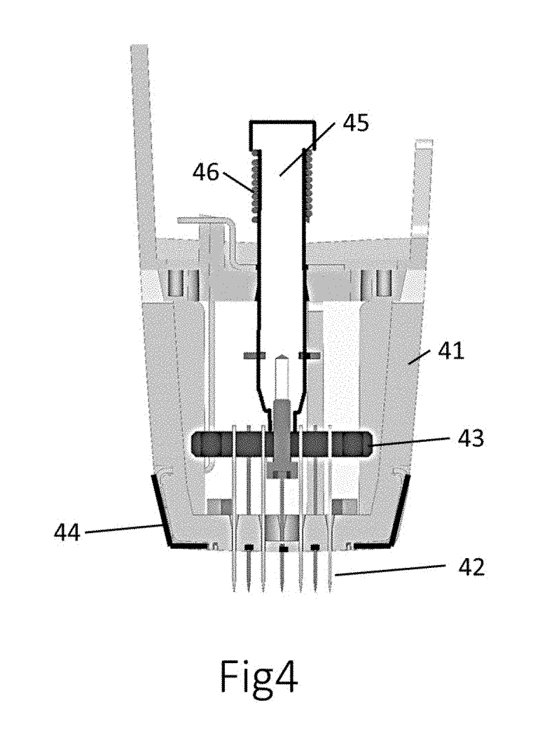

FIG. 4 shows a cross section of the disposable tip. An external electrode 44 is attached to a (e.g., plastic) housing 41. Needles 42 are assembled on a PCB 43 which is rigidly connected to a rod 45 which can move along the tip axes. A spring 46 acts to move needles out of the tissue after RF energy is delivered to the fat layer. This spring mechanism is a safety feature to keep needles inside the tip when the tip is not in use.

The device can be operated in two modes:

1. The needles are extended out of the tip prior to the treatment to a predetermined length and then the user applies tip with firm pressure to the treated area and applies RF energy. This method can be use when needle length does not exceed 3 mm.

2. The other method includes application of the hand piece with hidden (retracted) needles and then extending the needles out of the tip into the tissue to a predetermined depth at each pulse. The extending of the needles is synchronized with RF pulses. After RF delivery, the needles are retracted back into the tip.

After the needles 42 penetrate the tissue to the predetermined depth, the RF voltage is applied between the needles and the return electrode. RF energy per needle is high enough to create coagulation or ablation of the adipose tissue in the vicinity of the needles. After delivering the RF energy, the electromechanical mechanism releases the pressure allowing the spring 25 to retract the needles out of tissue. Penetration depth can be preprogrammed in the range of 0.6 mm up to 10 mm, without limitation.

RF energy delivered to the tissue depends on the number of needles and may be in the range of 0.1 J up to 30 J, without limitation.

RF pulse duration may be in the range of 1 ms and up to 3 sec, without limitation. The energy can be delivered as a single pulse or structured from the train of pulses.

Non-limiting parameters for the above described device are:

1. Number of conductive elements is in the range of 5 to 100

2. Shape of conductive elements is a sharp needle for deep treatment.

3. Length of needles is in the range of 0.6 mm to 10 mm.

4. Needles are partially coated with electrically isolating material and have electrically conductive ends to deliver RF energy into fat layer and avoid thermal damage of dermis.

5. RF voltage applied to the skin may be in the range of 10V up to 1000V RMS

6. Pulse repetition rate from 0.2 pps up 3 pps

1. A method for fractional thermal destruction of subcutaneous fat comprising:
applying a treatment tip to a skin surface;
inserting partially isolated needles from said treatment tip into tissue below the skin surface to place conductive tips of the needles into subcutaneous fat tissue;
delivering RF energy to the fat tissue through the conductive tips; and
maintaining the RF energy for a period of time to cause fat thermal destruction at said conductive tips.

2. The method according to claim 1, wherein the needles are inserted to a depth from 0.6 mm up to 10 mm.

3. The method according to claim 1, wherein the RF energy is applied between said needles and a return electrode applied to the skin surface.

4. The method according to claim 3, wherein the return electrode surrounds each needle.

5. The method according to claim 1, wherein the RF energy is delivered in pulses.

6. The method according to claim 1, wherein the RF energy has power from 1 W up 500 W.

7. The method according to claim 1, wherein the needles are moved manually.

8. The method according to claim 1, wherein the needles are moved by an electromechanical element.
